(19) United States
(12) Patent Application Publication
(10) Pub. No.: US 2012/0329831 A1
(43) Pub. Date: Dec. 27, 2012

(54) PHARMACEUTICAL COMPOSITION OF DONEPEZIL

(75) Inventors: Anuj Kumar FANDA, Ghaziabad (IN); Kumaravel VIVEK, Chennai (IN); Romi Barat SINGH, Varanasi (IN); Ajay Kumar SINGLA, Gurgaon (IN)

(73) Assignee: RANBAXY LABORATORIES LIMITED, New Delhi (IN)

(21) Appl. No.: 13/530,736

(22) Filed: Jun. 22, 2012

(30) Foreign Application Priority Data
Jun. 27, 2011 (IN) 1818/DEL/2011

(51) Int. Cl. A61K 31/445; A61P 25/28

(52) U.S. Cl. 514/319

(57) ABSTRACT

The present invention relates to a pharmaceutical composition of donepezil or pharmaceutically acceptable salts thereof comprising ethyl cellulose and methacrylic acid copolymer in a ratio of more than 1.3. Further, it relates to process for preparation of said composition.

### FIELD OF THE INVENTION

The present invention relates to a pharmaceutical composition of donepezil or pharmaceutically acceptable salts thereof comprising ethyl cellulose and methacrylic acid copolymer in a ratio of more than 1.3. Further, it relates to process for preparation of said composition.

### BACKGROUND OF THE INVENTION

Donepezil is a reversible inhibitor of the enzyme acetylcholinesterase. Its main therapeutic use is in the treatment of Alzheimer's disease.

The immediate-release donepezil results in a spike in the patient's blood plasma levels within 2 hours to 5 hours after administration of the drug. The initial spike in blood plasma levels may cause undesirable side effects in patients, such as anxiety, nightmares, insomnia, and gastrointestinal problems.

Currently, donepezil is being marketed under the trade name Aricept® by Eisai. Aricept® is available as immediate-release tablets and orally disintegrating tablets with a dose of 5 mg and 10 mg and is generally administered once per day. Eisai is also marketing 23 mg donepezil tablets in the United States.

Various attempts to prepare sustained-release formulation of donepezil have been reported.

U.S. Publication No. 2009/0208579 discloses a matrix type sustained-release preparation comprising a combination of an enteric polymer with a water-insoluble polymer.

U.S. Publications Nos. 2006/0280789 and 2007/0129402 disclose a pharmacokinetic profile of a sustained-release pharmaceutical formulation comprising a combination of an enteric polymer with a water-insoluble polymer.

U.S. Publication No. 2008/0213368 discloses a method for stabilizing a pharmaceutical composition comprising high molecular weight basic substance and donepezil by adding a high molecular weight acidic substance to said pharmaceutical composition; wherein said high molecular weight acidic substance includes enteric polymer and high molecular weight basic substance includes water-insoluble polymer.

The prior art formulation requires a combination of enteric polymer and ethyl cellulose for preparing a pharmaceutical composition of donepezil. Katikaneni et al., (International Journal of Pharmaceutics 117, p. 13-21 (1995)) report that some critical tableting properties of ethyl cellulose, for example tablet strength, are sensitive to press speed, particle size and the type of lubricant used and this introduces uncertainty to the tableting process.

The present inventors have surprisingly found that using a specific ratio of ethyl cellulose and methacrylic acid copolymer is critical to obtain a composition which has in vitro as well as in vivo profile comparable to the innovator, Aricept® 23 mg.

The present inventors have now developed an alternate pharmaceutical composition of donepezil comprising ethyl cellulose and methacrylic acid copolymer in a ratio greater than 1.3.

### SUMMARY OF THE INVENTION

Hence, according to one of the aspects, there is provided a pharmaceutical composition comprising:
a) donepezil or pharmaceutically acceptable salts thereof;
b) ethyl cellulose; and
c) methacrylic acid copolymer; wherein the ratio of ethyl cellulose to methacrylic acid copolymer is greater than 1.3.

According to another aspect, there is provided a pharmaceutical composition comprising:
a) donepezil or pharmaceutically acceptable salts thereof;
b) ethyl cellulose; and
c) methacrylic acid copolymer; wherein the ratio of ethyl cellulose to methacrylic acid copolymer is 1.3 to 2.0.

According to another aspect, there is provided a pharmaceutical composition comprising:
a) donepezil or pharmaceutically acceptable salts thereof;
b) ethyl cellulose; and
c) methacrylic acid copolymer selected from the group consisting of methacrylic acid-methyl methacrylate copolymer and methacrylic acid-ethyl acrylate copolymer; wherein the ratio of ethyl cellulose to methacrylic acid copolymer is 1.3 to 2.0.

According to another aspect, there is provided a pharmaceutical composition comprising:
a) donepezil or pharmaceutically acceptable salts thereof;
b) ethyl cellulose; and
c) methacrylic acid-ethyl acrylate copolymer; wherein the ratio of ethyl cellulose to methacrylic acid-ethyl acrylate copolymer is 1.3 to 2.0.

According to another aspect, there is provided a pharmaceutical composition comprising:
a) donepezil or pharmaceutically acceptable salts thereof;
b) ethyl cellulose; and
c) methacrylic acid copolymer; wherein the ratio of ethyl cellulose to methacrylic acid copolymer is 1.3 to 2.0, and the composition releases 40% to 60% of donepezil in 4 hours when measured in a USP type II dissolution apparatus, paddle rotating at 50 rpm, at a temperature of 37° C.±0.5° C., in 900 ml of pH 6.8 phosphate buffer.

According to another aspect, there is provided a pharmaceutical composition comprising:
a) donepezil or pharmaceutically acceptable salts thereof;
b) ethyl cellulose; and
c) methacrylic acid copolymer; wherein the ratio of ethyl cellulose to methacrylic acid copolymer is 1.3 to 2.0, and the composition releases 45% to 58% of donepezil in 4 hours when measured in a USP type II dissolution apparatus, paddle rotating at 50 rpm, at a temperature of 37° C.±0.5° C., in 900 ml of pH 6.8 phosphate buffer.

According to another aspect, there is provided a process for preparation of pharmaceutical composition comprising the steps of:
a) blending donepezil with ethyl cellulose, methacrylic acid copolymer, a binder and one or more pharmaceutically acceptable excipients;
b) granulating the blend of step a) with a binder solution;
c) lubricating the granules of step b); and
d) compressing the blend of step c) into a suitable size tablet.

### DETAILED DESCRIPTION OF THE INVENTION

The present inventors have found that the ratio of ethyl cellulose to methacrylic acid copolymer is critical for obtaining a composition which has a dissolution profile comparable to Aricept® 23 mg tablets. The ratio should be greater than 1.3, in particular 1.3 to 2.0. Also, the present inventors have determined that the 4 hour time point in the dissolution profile is critical, to have pharmacokinetic profile similar to Aricept® 23 mg tablets.

The term “pharmaceutically acceptable salts”, as used herein, includes organic or inorganic acid salts of donepezil, for example, hydrochlorides, sulfates, acetates, phosphates, carbonates, mesylates, tartrates, citrates and tosylates.

Donepezil may be present alone or in combination with other anti-dementia drugs such as NMDA receptor antagonists (e.g. memantine), choline uptake enhancers (e.g. MKC-231), somatostatin release enhancers (e.g. FK960), neurotransmitter regulators (e.g. nefiracetam), muscarinic M1 receptor agonists (e.g. talsaclidine), benzodiazepine receptor partial inverse agonists (e.g. S-8510), and acetylcholine/noradrenaline release enhancers (e.g. T-588, T-817MA).

Ethyl cellulose is available in various grades varying with respect to particle size, for example, fine particle size (FP) or standard or viscosity (7, 10, 50 and 100 cp). The amount of ethyl cellulose may vary from 15% to 40% by weight of the total composition.

“Methacrylic acid copolymer” includes methacrylic acid-methyl methacrylate copolymer (for example, Eudragit® L 100 or Eudragit® S 100) and methacrylic acid-ethyl acrylate (for example, Eudragit® L100-55). Also, the amount of methacrylic acid copolymer may vary from 10% to 30% by weight of the total composition.

“Pharmaceutical composition” as used herein may be in the form of tablets, granules or capsules. “Tablets” as used herein may be in the form of matrix type or coated type.

The pharmaceutical composition may further comprise other pharmaceutically acceptable excipients which include all excipients used in the art of manufacturing solid dosage forms. Examples include binders, diluents, lubricants/glidants, film-forming polymers and coloring agents.

Specific examples of binders include methyl cellulose, hydroxypropyl cellulose (HPC-L), methylcellulose, carboxymethyl cellulose sodium, hydroxypropyl methylcellulose, polyvinylpyrrolidone, and mixtures thereof.

Specific examples of diluents include lactose, calcium carbonate, calcium phosphate-dibasic, calcium phosphate-tribasic, calcium sulfate, cellulose-microcrystalline, cellulose-powdered, dextrates, dextrins, dextrose excipients, fructose, kaolin, lactitol, mannitol, sorbitol, starch, starch corn, sucrose, sugar compressible, sugar confectioners, and mixtures thereof.

The coloring agent may be selected from FDA approved colorants such as Iron Oxide, Lake of Tartrazine, Allura Red, Lake of Quinoline Yellow, Lake of Erythrosine, titanium dioxide, and mixtures thereof.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and mixtures thereof. It may be added intragranularly as well as extragranularly.

The pharmaceutical composition may further be coated with non-functional layers comprising film-forming polymers, if desired.

Examples of film-forming polymers such as polyvinyl pyrrolidone, hydroxypropyl methylcellulose and hydroxypropyl cellulose may be used. Alternatively, commercially available coating compositions comprising film-forming polymers marketed under various trade names, such as Opadry® may also be used for coating.

According to one embodiment, there is provided a process for the preparation of a pharmaceutical composition for oral administration, the process comprising the steps of:
a) blending donepezil with ethyl cellulose, Eudragit® and binder, and one or more pharmaceutically acceptable excipients;
b) granulating the blend of step a) with a binder solution;
c) lubricating the granules of step b); and
d) compressing the blend of step c) into a suitable size tablet.

Tablets may be prepared by direct compression method or granulation method. Granules can be prepared by dry granulation or wet granulation. Wet granulation may be carried out using granulating fluid or binder solution. The binder solution may comprise a suitable hydrophilic polymer dispersed or dissolved in a solvent. Dry granulation may be carried out by roller compaction or slugging.

The solvent used for granulation and coating may be selected from water, alcohols, for example, methyl alcohol, ethyl alcohol or isopropyl alcohol, acetone, and mixtures thereof.

The following examples illustrate the invention but do not limit the scope of the invention.

### EXAMPLES

### Example 1

| | Qty |
|---|---|
| Ingredients | (Mg/Tablet) |
| Donezepil Hydrochloride | 23.00 |
| Lactose Monohydrate | 100.00 |
| Eudragit ® L | 26.00 |
| Ethyl Cellulose | 44.00 |
| Hydroxypropyl Cellulose-L | 3.00 |
| Water | QS |
| Hydroxypropyl Cellulose-L | 3.00 |
| Magnesium Stearate | 3.00 |

### Process:

1) Donepezil hydrochloride, ethyl cellulose, Eudragit® L, lactose monohydrate and hydroxypropyl cellulose-L were blended together.
2) Second portion of hydroxypropyl cellulose-L was dissolved in water.
3) Blend of step 1) was granulated with solution of step 2).
4) Granules of step 3) were lubricated with magnesium stearate.
5) Blend of step 4) was compressed into a suitable size tablet.

### COMPARATIVE EXAMPLES

### Comparative Example 1

Comparative Example 1 has the same ratio of ethyl cellulose to Eudragit® as given in Example 31 of U.S. Publication 2006/0280789.

| | Qty |
|---|---|
| Ingredients | (mg/tablet) |
| Donezepil Hydrochloride | 23.00 |
| Lactose Monohydrate | 84.00 |
| Eudragit ® L | 42.00 |
| Ethyl Cellulose | 44.00 |
| Water | qs |
| Hydroxypropyl Cellulose-L | 6.00 |
| Magnesium Stearate | 3.00 |

### Process:

1) Donepezil hydrochloride, ethyl cellulose, Eudragit® L and lactose monohydrate were blended together.
2) Hydroxypropyl cellulose-L was dissolved in water.
3) Blend of step 1) was granulated with solution of step 2).
4) Granules of step 3) were lubricated with magnesium stearate.
5) Blend of step 4) was compressed into a suitable size tablet.

### Dissolution Studies:

The dissolution tests were carried out using tablets prepared in Example 1 and Comparative Example 1 as well as Aricept® 23 mg.

The dissolution was carried out in USP type II apparatus, paddle rotating at 50 rpm, at a temperature of 37° C.±0.5° C., in 900 ml of pH 6.8 phosphate buffer with alternate sinkers. The dissolution rate was calculated from concentration of donepezil hydrochloride in the sample solutions collected at different dissolution time and analyzed by an HPLC method using Kromasil C18 column (5 μm) and mobile phase comprising mixture of buffer (pH 2.2) and methanol (a mixture of buffer and methanol in the ratio of 50:50) and was measured at 268 nm by UV detector.

TABLE 1

| | % Drug Released (Donepezil) | | |
|---|---|---|---|
| | in 900 ml of Phosphate Buffer | | |
| Time | | Comparative | |
| (hrs) | Example 1 | Example 1 | Aricept ® 23 mg |
| 1 | 24 | 12 | 21 |
| 2 | 34 | 17 | 31 |
| 4 | 51 | 25 | 48 |
| 6 | 67 | 37 | 68 |
| 8 | 84 | 48 | 85 |
| 12 | 100 | 65 | 97 |

The results of the dissolution tests are shown in Table 1. It is evident that Example 1 provides the desired release profile.

1. A pharmaceutical composition comprising:
a) donepezil or pharmaceutically acceptable salts thereof;
b) ethyl cellulose; and
c) methacrylic acid copolymer selected from the group consisting of methacrylic acid-methyl methacrylate copolymer and methacrylic acid-ethyl acrylate copolymer; wherein the ratio of ethyl cellulose to methacrylic acid copolymer is 1.3 to 2.0.

2. The pharmaceutical composition according to claim 1 releases 40% to 60% of donepezil in 4 hours when measured in a USP type II dissolution apparatus with paddle rotating at 50 rpm in 900 ml of pH 6.8 phosphate buffer.

3. The pharmaceutical composition according to claim 1, wherein said methacrylic acid copolymer is methacrylic acid ethyl acrylate copolymer.

4. The pharmaceutical composition according to claim 1 further comprises pharmaceutically acceptable excipients selected from the group consisting of binders, diluents, lubricants, film-forming polymers and coloring agents.

5. The pharmaceutical composition according to claim 1, wherein said composition is in the form of tablets.

6. The pharmaceutical composition according to claim 5, wherein said tablets are matrix type tablets.

7. The pharmaceutical composition according to claim 5, wherein said tablet is prepared by a process comprising the steps of:
a. blending donepezil with ethyl cellulose, methacrylic acid copolymer, a binder and one or more pharmaceutically acceptable excipients;
b. granulating the blend of step a) with a binder solution;
c. lubricating the granules of step b); and
d. compressing the blend of step c) into a suitable size tablet.

8. The pharmaceutical composition according to claim 7, wherein the binder in step a) and step b) are the same.

9. The pharmaceutical composition according to claim 7, wherein the binder in step a) and step b) are different.

10. The pharmaceutical composition according to claim 7, wherein said binder is selected from the group consisting of methyl cellulose, hydroxypropyl cellulose, methylcellulose, carboxymethyl cellulose sodium, hydroxypropyl methylcellulose and polyvinylpyrrolidone.
